(19) United States
(12) Patent Application Publication
(10) Pub. No.: US 2004/0191763 A1
(43) Pub. Date: Sep. 30, 2004

(54) HBV mutations associated with reduced susceptibility to adefovir

(75) Inventors: William E. Delaney, San Mateo, CA (US); Xiaofeng Xiong, Cupertino, CA (US)

Correspondence Address: GILEAD SCIENCES INC, 333 LAKESIDE DR, FOSTER CITY, CA, 94404, US

(73) Assignee: GILEAD SCIENCES, INC.

(21) Appl. No.: 10/678,454

(22) Filed: Oct. 1, 2003

Related U.S. Application Data
(60) Provisional application No. 60/415,018, filed on Oct. 1, 2002.

(51) Int. Cl. C12Q 1/70; A01K 67/00; C07H 21/04; C12N 7/00; C07K 14/02; C12N 15/86

(52) U.S. Cl. 435/5; 435/69.3; 435/320.1; 435/325; 435/235.1; 435/456; 536/23.72; 800/9

(57) ABSTRACT

Applicants have identified 5 mutants associated with hepatitis B virus resistance to adefovir, a nucleotide analogue antiviral drug widely employed in the therapy of hepatitis B. In accord with this invention, reverse transcriptase mutants rtN236T, rtA181V, rtA181T and their corresponding surface antigen mutants sL173F and sL172trunc are provided. The mutant proteins, antibodies thereto and nucleic acids encoding the mutants have diagnostic value in monitoring and adjusting patient therapy with adefovir and in the therapy of patients infected with the mutants.

Before the advent of adefovir (ADV) therapy, lamivudine and interferon were the only two approved therapies for the treatment of chronic hepatitis B virus infection. Interferon therapy is associated with serious side effects including flu-like symptoms, fever, and depression. Long-term lamivudine therapy is limited by the high incidence and rapid onset of resistance that occurred in 24% of patients at one year and 70% of patients after four years of therapy [1]. Lamivudine resistance is predominately associated with mutations (rtM204V or rtM204I) in the YMDD motif in the C domain of the HBV polymerase (reverse transcriptase, or “rt”). The consensus nomenclature of HBV polymerase mutations is used throughout this report [2]. The rtL180M and rtV173L mutations in the B domain of HBV polymerase were also frequently observed in conjunction with the YMDD mutations in lamivudine-resistant HBV. The B domain mutations did not confer significant resistance to lamivudine on their own. Instead, these mutations appeared to enhance replication fitness of the YMDD mutant HBV [3]. Other HBV polymerase mutations were reported at much lower frequencies in patients receiving lamivudine. These low frequency mutations have not been established as lamivudine resistance mutations.

Adefovir, an acyclic analog of adenosine monophosphate, belongs to a new class of nucleotide antivirals. Adefovir has demonstrated potent activity against wild-type and lamivudine-resistant HBV in vitro and in vivo. In addition, it also showed activity against retroviruses and herpesviruses in vitro. Adefovir dipivoxil, an oral prodrug of adefovir, enhances the bioavailability of adefovir in patients. In cells, adefovir requires two phosphorylation steps to convert to the active metabolite adefovir diphosphate, which is a potent competitive inhibitor of HBV polymerase with respect to the natural substrate dATP and functions as a chain-terminator of viral replication. The inhibition constant (Kᵢ) for adefovir diphosphate was 0.1 μM in an enzymatic assay using recombinant HBV polymerase [4]. Adefovir has demonstrated in vitro antiviral activity against human HBV, duck HBV (DHBV), and woodchuck hepatitis virus (WHV) in cell culture models with IC₅₀ values in the range of 0.2 to 1.2 μM [5-10].

Two Phase 3 clinical studies demonstrated the anti-HBV activity of ADV at 10 mg daily in chronic hepatitis B patients. The median serum HBV DNA levels declined by 3.5 and 3.9 log₁₀ copies/mL from baseline to week 48 in the ADV 10 mg dose groups in HBeAg positive (Study GS-98-437) and in HBeAg negative (Study GS-98-438) chronic hepatitis B patients, respectively [11, 12]. Additional virological and clinical benefits were observed with extended ADV therapy up to 96 weeks in the HBeAg negative patients in study GS-98-438 [13].

In contrast to the high incidence of resistance in patients receiving lamivudine therapy, no adefovir resistance mutations were identified in chronic hepatitis B patients in the two Phase 3 ADV clinical studies after 48 weeks of ADV therapy [14]. In addition, 48 weeks of ADV treatment did not lead to selection of adefovir-resistant HBV in HIV/HBV co-infected and post-liver transplantation patients with lamivudine-resistant HBV [15, 16].

### SUMMARY OF THE INVENTION

We have now identified five HBV rt and HBsAg mutations associated with adefovir resistance: rtN236T, rtA181V, rtA181T, surface antigen (“sAg”) L173F and sAg which is terminated immediately N-terminal to residue L172 (hereafter “sL172trunc”). The sAg and rt position 181 mutations are related in that the open reading frame for rt and sAg overlap in part. The rtA181V and rtA181T mutants correspond respectively to the sL173F and sL172trunc mutants (the latter resulting from substitution of a stop codon into the sAg reading frame). The HbsAg sequence before the introduced stop codon is SVRFS, with the C-terminal serine residue being at sAg position 171.

While rtN236T is the only mutation presently associated with clinical manifestations of resistance, e.g., viral load rebound, the remaining mutations have value in diagnosis and therapy of HBV infection, as do their antibodies and nucleic acids encoding the mutants. Accordingly, embodiments of the invention include isolated nucleic acid encoding hepatitis B virus rtN236T, rtA181V, rtA181T, sL173F and/or sL172trunc; nucleic acid encoding hepatitis virus rtN236T, rtA181V, rtA181T, sL173F and/or sL172trunc which is fused with heterologous nudeic acid; isolated infectious hepatitis virus comprising nucleic acids encoding one or more of the mutants; vectors comprising nucleic acid encoding one or more of the mutants; host cells transformed with the vectors and methods for culturing such cells and recovering mutant polypeptide therefrom.

Animal models of infection which contain one or more of these mutants are another embodiment of the invention. WHV and DHBV are known models, as noted above. In an embodiment of the invention, corresponding mutations are introduced into WHV or DHBV and permissive hosts infected with the mutant-bearing virus. The woodchuck and duck mutations corresponding to rtN236T are, respectively, N620T and N544T.

In other embodiments of the invention the mutant rt or sAg polypeptides or their fragments are provided in isolated form, fused with heterologous polypeptides, bound to a detectable label or to an insoluble substance or are combined in a composition with a pharmaceutically acceptable excipient.

In further embodiments, antibodies are provided that are capable of specifically binding one or more of the rt or sAg mutant polypeptides. These antibodies also are provided in isolated form, fused with heterologous polypeptides, bound to a detectable label or to an insoluble substance or are combined in a composition with a pharmaceutically acceptable excipient.

In another embodiment of the invention, the mutant proteins or nucleic acid are assayed using conventional methods and the results used to guide clinical decision making. In particular, the mutants (especially the rtN236T mutant) are monitored and, upon emergence, an additional therapeutic agent which does not cross-resist with adefovir is added to the regimen. Alternatively, such agents are employed in prophylaxis to suppress or prevent emergence of the mutants in vivo.

In another embodiment, a PCR kit is provided that comprises primers capable of amplifying a hepatitis nucleic acid encoding at least one of the mutants of this invention.

Other embodiments of the invention will be apparent from the disclosure and claims following.

### DETAILED DESCRIPTION OF THE INVENTION

Adefovir resistance surveillance was performed in a blinded manner in HBeAg negative chronic hepatitis B patients treated for 96 weeks in a Phase 3 double-blind, randomized, placebo-controlled clinical study (GS-98-438) of adefovir dipivoxil 10 mg (ADV). The analysis included 79 patients who received ADV 10 mg daily for the first 48 weeks and were randomized to also receive ADV 10 mg during the second 48 weeks.

The reverse transcriptase (RT) domain of the HBV polymerase gene (rt1 to rt344) was sequenced for HBV DNA extracted from baseline and week 96 serum samples (or the last on-study sample) for the 79 patients treated continuously with ADV for 96 weeks if the samples had detectable serum HBV DNA (≧1000 copies/mL by Roche Amplicor™ PCR assay) at the time points tested.

Of the 79 patients, paired baseline and week 96 (or last on-study sample) sequences were obtained for 20 patients. Paired genotypes were not obtained for 58 of the 79 ADV-treated patients with serum HBV DNA levels <1000 copies/mL at week 96 (or the last on-study visit). One additional patient was not genotyped because of unsuccessful PCR amplification associated with a low serum HBV DNA (1457 copies/mL) for the week 96 sample.

Novel conserved site mutations emerged in five patients. The rtN236T mutation was observed in two patients (0454-2506 and 0626-1537). In vitro phenotypic analyses of patient-derived HBV clones carrying rtN236T showed a 7- to 14-fold reduced susceptibility to adefovir. Patient 0454-2506 also developed another conserved site mutation rtA181T, which did not confer resistance to adefovir in vitro. The emergence of the rtN236T mutation was associated with serum HBV DNA rebound (defined as a confirmed ≧1.0 log₁₀ increase in HBV DNA from an on-treatment nadir at two consecutive visits while on ADV therapy) in both patients. The rtN236T mutant remained susceptible to lamivudine (IC₅₀ changed by ≦3.5-fold) in vitro. Serum HBV DNA suppression was observed clinically for one patient (0454-2506) who switched to lamivudine therapy.

The rtA181V mutation occurred in two patients (0624-1517 and 0624-1564). Patient-derived HBV clones containing the rtA181V mutation demonstrated 2.5- to 3-fold reduced susceptibility to adefovir in vitro. Only one patient with the rtA181V mutation had serum HBV DNA rebound. The other patient with the same mutation maintained full suppression of serum HBV DNA (<1,000 copies/mL after week 112). The association of the rtA181V mutation with resistance to ADV remains unclear.

A fifth patient (0370-3503) developed two conserved site mutations rtK241E and rtK318Q. This double mutation was not associated with resistance to adefovir in vitro nor associated with serum HBV DNA rebound in vivo.

The adefovir resistance mutation rtN236T demonstrated moderate cross-resistance (4- to 8-fold) to acyclic nucleotides tenofovir and MCC-478 in vitro. The rtN236T mutant remained susceptible to L-nucleoside analogs such as lamivudine and L-dT as well as carbocyclic nucleoside analog entecavir in vitro.

The novel rtN236T mutation conferred reduced susceptibility to adefovir and serum HBV DNA rebound was identified in 2 of 79 (2.5%) presumed precore mutant chronic hepatitis patients taking ADV for 96 weeks. This mutation remained susceptible to lamivudine in vitro and in vivo.

The novel hepatitis B virus rt and sAg compositions of this invention are readily identified by methods heretofore known per se in the art. Typically, one assays for the mutant protein, or nucleic acid (DNA or RNA) encoding same. Suitable methods include, for example, 1) direct DNA sequencing of PCR-amplified products, 2) sequencing of cloned viral DNA, 3) tests using restriction fragment length polymorphism (RFLP), 4) assays based on the hybridization of DNA fragments by means of nucleic acid probes (PCR/real-time PCR including differential detection of mutant with nucleotide probes, or by melting curve analyses of PCR products, and line probe assay (immobilized reverse hybridization probes), 5) matrix-assisted laser desorption ionization time-of-flight mass spectrometry (MALDI-TOF MS) (Ding et al., PNAS 100(6):3059 Mar. 18, 2003), 6) oligonucleotide microarrays (DNA chips), 7) Linear signal amplification (INVADER assay) (Cooksey et al., Antimicrobial Agents and Chemotherapy 44(5):1296 May 2000), 8) serial invasive signal amplification reaction (Ding et al., PNAS 97(15):8272, Jul. 18,2000), and 9) methods of immunological detection such as and ELISA or radioimmunoassay. Neither the method by which the variants are detected nor the form in which they are detected is critical to the practice of this invention.

“Isolated” when used in reference to the protein or nucleic acid variants of this invention means the protein or nucleic acid is not present in its native environment. Typically, this means the mutant protein or nucleic acid is free of at least one of the viral or host proteins or nucleic acids with which each is ordinarily associated. In general, isolated proteins are nucleic acids are present in an in vitro environment. “Isolated” does not mean that the protein or nucleic acid must be purified or homogeneous, although such preparations do fall within the scope of the term. “Isolated” simply means raised to a degree of purity to the extent required to exclude products of nature and accidental anticipations from the scope of the claims.

Proteins of this invention need not be purified at all to be “isolated”. For example, a cell culture of recombinant cells expressing a mutant protein of this invention is itself an “isolated” form of the mutant protein. In general, of course, optimal results are obtained with protein that has been more than simply placed into an environment which is distinct from that of its natural occurrence. Thus, protein optionally is purified (either from cultured or recovered hepatitis virus or from recombinant cell culture of heterologous transformants). Typically, the proteins will be purified to a single band in gel chromatography, but other methods are freely employed. Suitable methods have been used before for the wild type proteins. In addition, antibodies capable of binding the proteins of this invention are employed in immunoaffinity purification of the proteins. These methods are known per se.

Nucleic acids encoding the variants of this invention optionally are RNA or DNA, which optionally vary in sequence length and the selection of bases flanking the mutant residue codon. The length of the nucleic acid is not critical. Sufficient nudeic acid need only be present to provide novelty and utility for the sequence encoding the variant, but otherwise the length of the sequence flanking the selected codon is not important. Typically the length of the sequence (including the variant codon) will be any integer from with the range of 9 to 200 bp, usually about 12 to 30 bp and most typically 15 to 25 bp. Also included are sequences sufficiently long to encode the entire variants and their enzmatically or antigenically active fragments further described below.

The sequence flanking the variant codon also is not critical provided that it is recognized to be a hepatitis B virus sequence for the purpose intended. This virus is highly polymorphic. Considerable sequence variation exists within its genome, although some regions are more conserved than others. Genbank contains at least 70 HBV reference sequences alone, and more are being added as time goes on. Thus the nucleic acid sequences flanking the variant sites vary considerably even in the naturally occurring sequences. In addition, further or different variations in sequence or codon choice optionally are introduced into any of these native sequences (for example to provide novel restriction sites). The resulting sequence need only be capable of functioning in a diagnostic assay for the natural variant or in an expression system to produce protein having the intended diagnostic or therapeutic use. More nucleic acid sequence variation is accommodated in connection with expression of the variant proteins because any codons for the particular amino acid residue can be employed. Flanking sequence also is varied to allow for fusion to heterologous nucleic acids (as in construction of expression or cloning vectors, in diagnostic assay constructs heretofore known, and for expression of fusion proteins). In regard to the diagnostic assays for variant nucleic acids, one or more native base pairs are optionally substituted (or one is inserted or deleted) so long as the resulting sequence remains capable of acting as, for instance, a PCR primer or hybridization reagent. Determination of which nucleic acid sequence variants will be useful is simply a matter of routine experimentation and is well within the skill of the ordinary artisan.

The term “nucleic acid” is not intended to imply a size limitation. For the purposes herein it includes oligonucleotides or other short length sequences, for example probes or primers.

Polymerase Chain Reaction (PCR) assays are readily employed to detect the mutant nucleic acids herein. Such PCR methods preferably use at least one amplification primer that include the mutant codon or the complementary sequence. Thus, in a PCR kit, primers are supplied that are capable of amplifying the nucleic acid encoding the mutant, whether or not the sequences are novel.

Also included within the scope of the invention are nucleic acid sequences complementary to the foregoing nucleic acids, vectors containing variant nucleic acid or its complement, host cells transformed with such vectors (together with cell cultures thereof), and methods for recombinant expression of the variant proteins. Methods for recombinant expression include expressing the variant rt in transformed cell culture (human, animal or microbial host cells infected with virus bearing the mutant or transformed by a vector containing the its nucleic acid). Methods for recombinant expression are known per se, and many of them have been used heretofore in the expression of wild type rt and sAg. Any of these are suitable for use herein.

Nucleic acids of this invention include nucleic acids that hybridize to the naturally occurring sequences. These may be full length rt or sAg sequences, or any fragments thereof having the desired character. A hybridizing nucleic acid is one that binds to the target sequence under stringent conditions (see U.S. Pat. No. 6,110,721). Other nucleic acids of this invention are defined by their degree of sequence homology to a native sequence. Typically, this could be 80, 85, 90, 95 or 99% homologous to the hepatitis B virus sequence bearing one of the mutants herein, but as a practical matter primer or probe homology is defined functionally. The probe or primer need only bind to the target sequence under standard commercial assay conditions with sufficient specificity as to exclude the wild type or unmutated hepatitis virus in the patient or population concerned. Standard commercial assay conditions will of course vary from assay system to assay system, and the sequence homologies permitted will vary accordingly. Optimal probes and primers will use the codon choice for the mutant residues found within the patient population (and the assays may in fact use a plurality of probes or primers representing variation in the population). Determination of the suitable sequences then is simply a matter of routine experimentation well within the skill in the art.

Also useful are animal models of the resistant mutations of this invention. These are obtained by introducing the appropriate mutations into the position 236 and/or 181 correspondent positions of duck hepatitis B or woodchuck hepatitis virus. These are used to infect the permissive hosts and used to study the effect of drug combinations or other research as will be apparent to the ordinary artisan. The methods described in this paragraph are known per se and are not the invention herein. Their practice is well within the skill of the ordinary artisan.

It is possible that accidental anticipations of the sequence found within shorter length sequences do exist, i.e., the same sequence of base pairs found in 15 bp of variant nucleic acid about the site of mutation (or its complement, considering orientation) may also exist in an unrelated gene or known fragment thereof. In such instances, of course, the additional flanking sequences will be entirely distinct from hepatitis virus, but overlap could exist if the sequence that is being compared is sufficiently short. Accordingly the invention excludes any oligonucleotide or nucleic acid prior to the effective date hereof having an identical sequence to the mutant sequences of this invention, and optionally excludes all such sequences which while not identical bind to a mutant sequence hereof under stringent hybridization conditions (as defined in U.S. Pat. No. 6,110,721). These prior art sequences are readily identified by searching the GenBank database, and they are expressly incorporated by reference.

In an important embodiment of the invention, the rtN236T mutation is assayed to monitor patients for emergence of adefovir resistance. Similarly, it is useful to monitor rtA181V. If these mutations appear in a patient under treatment with adefovir, clinical intevention may be in order, for instance coadministering a supplemental non-cross reactive therapeutic agent along with adefovir. These agents include for example entecavir, L-dT, MCC-478, FTC, L-dC, L-FMAU, L-Fd4C, Lamivudine and tenofovir. Others are readily identified by the method set forth below. The clinical doses of these agents are known or could be readily deduced from available information by skilled clinicians, as would the appropriate prodrug forms of the agents (such as tenofovir DF).

HBV rtA181T, rtA181V, rtN236T, sL173F and sL172trunc have a variety of uses. For example, they are used as immunogens to raise antibodies. The variant polypeptides also are useful as immunomodulators per se or to raise antibodies passive immune treatment of hepatitis B infection. The mutant proteins are isolated from HBV or produced in recombinant cell culture, and are suitably used as an antigen to raise antibodies or as an immunomodulator, e.g. vaccine. The variant sAg also is useful as a reagent in immunoassays for sL173F (and therefore, by extrapolation, rtA181V). Similarly, sL172trunc is used for the same general purpose, but detects rtA181T.

The polypeptides of this invention include full length hepatitis B sAg or rt, fragments thereof comprising at least the mutant residue or site, and/or either of these fused to a heterologous polypeptide. “Heterologous” whether defining nucleic acid or proteins sequence means not the same as the native or known flanking sequences. Heterologous sequences include other HBV, human, animal or microbial sequences, polyHis or other affinity tags, or entirely fabricated sequences. Fragments typically will include the variant residue plus at least about 4 total flanking residues apportioned to either or both flanks of the mutant residue, usually 10 to 20 residues in total. “Protein” and “polypeptide” are used herein without any inference of size. The fragments have a size sufficient to be immunologically active, i.e., they will be sufficiently immunogenic (alone or fused to an immunogenic protein) at least in animals, typically mice, so as to produce an antibody which (a) cross-reacts with the native, full length variant polypeptide, and/or (b) cross-reacts with an antibody raised against full length variant. The degree of cross-reactivity typically is sufficient to enable the fragment to perform in an immunoassay for the mutant, or as an immunogen in raising antibodies (as vaccines, in humans) that cross-react with the native full length rt or sAg.

Immunogenic preparations of the proteins of this invention optionally are formulated with an immune adjuvant, known per se, to enhance the response.

The variant rt or sAg optionally are bound to a detectable label. Such labeled protein typically is used in diagnostic assays. The antigens also are useful when bound to an insoluble substance (e.g., Sepharose or other matrix) for absorbing labeled antibody in diagnostic assays or in preparative methods for purifying the antibody. Such methods are known per se. In short, the variant proteins are used to produce reagents in conventional fashion, or assayed in the same fashion as other proteins using known methods, as in any therapeutically or diagnostically significant protein.

Antibodies capable of binding to the variant rts or surface antigen are useful in therapeutics and in diagnostic assays. These antibodies optionally are human antibodies or humanized antibodies (made by methods known per se), or are monoclonal murine antibodies. The origin is not important unless the antibody is to be use in passive immunization (as with the sAg variants herein) of human patients, in which case human or humanized antibodies are desired to prevent immune reactions to the therapeutic.

Antibody directed against any one or more of the rt or sAg variants optionally is labelled, e.g., with a radioisotope or an enzyme, or is bound to an insoluble substance, generally for use in immunoassays. In another embodiment, antibodies of this invention (in the form of a pharmaceutically acceptable preparation) are useful in passive immunization, e.g., against the surface antigen variants (and by implication HBV bearing the rt181 mutants).

### Clinical Study Design

Study GS-98-438 is a randomized, double-blind, placebo-controlled Phase 3 clinical study of the safety and efficacy of ADV for the treatment of patients with HBeAg negative/anti-HBe positive/HBV DNA positive chronic hepatitis B. A total of 184 patients received at least one dose of study medication during the first 48 weeks (n=61 and 123 for the placebo and ADV 10 mg groups, respectively). During the second 48 weeks, all placebo patients switched to ADV 10 mg daily while the ADV-treated patients were re-randomized to either continue ADV or receive placebo at a 2:1 ratio. Accordingly, all patients who received at least one dose of study drug during the second 48 weeks are classified into three groups: PLB-ADV (n=60), ADV-ADV (n=79), and ADV-PLB (n=40). Baseline disease characteristics and demographics are summarized in Table 1.

TABLE 1
Baseline Disease Characteristics and Demographics
for ITT Population

| | Treatment Received | | | |
|---|---|---|---|---|
| | PLB- | ADV 10 mg- | ADV 10 mg- | |
| | ADV 10 mg | ADV 10 mg | Placebo | Total |
| | (n = 60) | (n = 79) | (n = 40) | (n = 179) |
| Median HBV | 7.1 | 7.1 | 0 7.2 | 7.1 |
| DNA (Log₁₀ | | | | |
| copies/mL) | | | | |
| Median ALT- | 2.4 | 2.3 | 2.1 | 2.3 |
| Multiples of | | | | |
| the Upper | | | | |
| Limit of | | | | |
| Normal | | | | |
| Median Age | 46 | 47 | 47 | 47 |
| Sex | | | | |
| Male | 83% | 82% | 83% | 83% |
| Race | | | | |
| White | 39 (65%) | 55 (70%) | 26 (65%) | 120 (67%) |
| Asian | 20 (33%) | 20 (25%) | 13 (33%) | 53 (30%) |
| Black | 1 (2%) | 4 (5%) | 1 (3%) | 6 (3%) |

### Virology Substudy

A virology substudy of Study GS-98-438 included all patients in the ADV 10 mg-ADV 10 mg group (n=79). The RT domain of the HBV polymerase gene from banked serum samples from these patients was genotypically analyzed at baseline, and either at week 96, or upon early termination during the second 48 weeks. In vitro phenotypic analyses of adefovir susceptibility were performed for patient-derived HBV clones if the patient had an emerging amino acid mutation at a conserved residue of HBV polymerase.

### Genotypic Analyses

Sample Inclusion Criteria

Genotypic analyses were performed for baseline, and either for week 96, or for the last on-drug serum samples (for patients who withdrew prior to week 96 ) that had a serum HBV DNA of ≧1,000 copies/mL as determined by the Roche Amplicor™ Monitor PCR Assay. If the week 96 serum HBV DNA value was not available for a patient, the closest serum HBV DNA value prior to week 96 was used. All week 96 and the last on-drug samples are referred to as week 96 samples hereafter. Note that Roche Molecular Systems raised the lower limit of quantification of the HBV DNA PCR assay from 400 copies/mL to 1,000 copies/mL after Gilead completed the week 48 analyses for study 438.

Genotyping Methods

Focus Technologies Inc. (Cypress, Calif.) was the designated reference laboratory for all HBV sequencing for Study GS-98-438. Briefly, HBV DNA was isolated from clinical serum specimens and amplified by PCR. The positive and negative strands of the HBV polymerase gene spanning the pol/RT domain (amino acids rt1 to rt344) were sequenced using 5 or 6 standard sequencing primers. Sequences were resolved on an automated DNA sequencer (ABI Prism 377, ABI, Foster City, Calif.). Based on plasmid mixing experiments, a mixture of wild-type and mutant nucleotides could be detected when either was present in the population at a frequency of ≧30%. Contiguous HBV sequences were assembled from the sequences of all samples using Autoassembler 2.0 (ABI).

The contiguous sequences for all samples were sent to Gilead Sciences for identification of HBV polymerase mutations. All data were received in the form of an electronic database.

Analyses of Sequencing Data

The contiguous nucleotide sequences (provided by Focus Technologies) for baseline and week 96 HBV samples from the same patient were aligned using the MegAlign sequence alignment program (DNAStar, Madison, Wis.). Amino acids present in the week 96 sample but not in the baseline sample for a patient were documented as emerging mutations. If there were no emerging amino acid mutations in a patient, the patient was documented as “no mutation”.

HBV RT domain sequences from 70 HBV isolates in GenBank and from 698 baseline HBV isolates in studies 437 and 438 were used as reference sequences to define conserved sites. A conserved site is defined as an amino acid residue that is unchanged either among the 70 Genbank HBV sequences or among the 698 baseline HBV sequences from studies 437 and 438. All other locations were considered to be polymorphic sites in HBV polymerase. Amino acid mutations emerging during the trial at polymorphic sites of the HBV polymerase were defined as polymorphic site mutations and those occurring at conserved sites were defined as conserved site mutations.

### In Vitro Phenotypic Analyses of Patient-Derived HBV Clones

All conserved site substitutions were evaluated for their effects on adefovir susceptibility using a novel approach to generate full-length patient-derived HBV clones. Briefly, viral DNA was extracted from patient serum and whole HBV genomes (3.2 kilobase) were PCR amplified using primers P1 (5′-CCG GAA AGC TTG AGC TCT TCT TTT TCA CCT CTG CCT AAT CA-3′) and P2 (5′-CCG GAA AGC TTG AGC TCT TCA AAA AGT TGC ATG GTG CTG G-3′). Full-length viral genomes were cloned into the lethal selection vector PCAP^{s} at a Mlu Nl site through blunt-end ligation (PCR Cloning Kit, Roche) and then subcloned into plasmid pHY106, a pBluescript KS (+)-derived plasmid containing a CMV promoter and the minimal 5′ and 3′ HBV sequence necessary (approximately 180 total bases) for viral replication after the insertion of a genome-length clinical HBV isolate. Drug susceptibility of patient-derived clones was analyzed by transient transfection into HepG2 cells. Transfected cells were treated with various concentrations of adefovir or lamivudine for 7 days and the amounts of intracellular replicating virus DNA were then quantified by Southern blotting to determine adefovir sensitivity.

### Emerging HBV Polymerase Mutations

Serum Samples Analyzed

Genotypic analyses were performed for all 79 baseline samples (Table 2). Twenty of the 79 week 96 samples were also genotypically analyzed. Two of the 20 patients with paired baseline and week 96 HBV genotyping data (0470-5514 and 0511-4509) had undetectable serum HBV DNA by the Roche Amplicor™ Monitor PCR assay at the last visits (week 92) during the blinded phase (prior to the open-label phase) of study 438. However, the week 96 samples from these two patients had detectable serum HBV DNA by the PCR assay and thus were genotypically analyzed in this virology substudy.

Paired HBV genotypes were not obtained for the remaining 59 patients. Fifty-eight of the 59 patients had undetectable serum HBV DNA levels (<1000 copies/mL) at week 96. One additional patient was not genotyped because of unsuccessful PCR amplification associated with a low serum HBV DNA (1457 copies/mL) for the week 96 sample (Table 2). These 59 patients were presumed not to be harboring resistant HBV strains since the serum HBV DNA levels were undetectable (or near undetectable) using the most sensitive commercial assay. The serum HBV DNA levels in these patients were well below the threshold of 100,000 copies/mL for serum HBV DNA that had previously been proposed as clinically significant [17].

TABLE 2
Genotypic Analysis Summary of Patients Who Received
Adefovir Dipivoxil for 96 Weeks in Study 438

| | Number of |
|---|---|
| | Patients |
| Total Number of Patients Included | 79 |
| for HBV Genotyping | |
| Baseline Samples | |
| Genotyped | 79 |
| Week 96 Samples¹ | |
| Genotyped | 20 |
| Not Genotyped | 59 |
| PCR negative | 1 |
| HBV DNA < 1000 copies/mL | 58 |

¹Week 96 samples or the last on-therapy samples.

Emerging HBV Polymerase Mutations

Of the 20 patients with both baseline and week 96 genotyping data, eight patients had at least one emerging amino acid mutation in the pol/RT domain of HBV polymerase at week 96. A total of 21 mutations were observed in these eight patients (Table 3). The majority of these mutations (14/21, 67%) occurred at polymorphic sites in the HBV polymerase. Since polymorphic mutations naturally exist in HBV of untreated patients, these mutations are unlikely to be associated with adefovir resistance. Such mutations, however, are present in HBV sequences and therefore have use in the diagnosis of HBV by immunological or nucleic acid-based methods. In addition, all three patients (0624-1528, 0624-1529, and 0624-1531) with only polymorphic mutations did not experience serum HBV DNA rebound through 96 weeks of ADV therapy, further suggesting that these polymorphic mutations were not associated with adefovir resistance.

TABLE 3
Emerging Mutations in HBV Reverse Transcriptase
Domain in Patients Who Received 96 Weeks of
Adefovir Dipivoxil in Study 438

| | Emerging Mutation | |
|---|---|---|
| Patient ID | in HBV RT at Week | Location in HBV RT |
| 0370-3503 | rtK241E¹ | D domain, conserved site |
| | rtH267Q | Downstream of E domain |
| | rtK318Q | Downstream of E domain, |
| | | conserved site |
| 0454-2506 | rtV134D/V | Inter A and B domains |
| | rtL145L/M | Inter A and B domains |
| | rtF151F/Y | Inter A and B domains |
| | rtA181A/T | B domain, conserved site |
| | rtN236T | D domain, conserved site |
| 0624-1517 | rtA181A/V | B domain, conserved site |
| | rtF221F/Y | Inter C and D domains |
| 0624-1528 | rtC/Y135N/Y | Inter A and B domains |
| | rtA/V214A/P | Inter C and D domains |
| | rtI266T | Downstream of E domain |
| 0624-1529 | rtI16I/T | Inter A and B domains |
| | rtH126H/R | Inter A and B domains |
| 0624-1531 | rtS256S/C | E domain |
| 0624-1564 | rtA181V | B domain, conserved site |
| 0626-1537 | rtN53D | Inter F and A domains |
| | rtY124H | Inter A and B domains |
| | rtN236T | D domain, conserved site |
| | rtN238T | D domain |

¹All conserved site mutations are in bold type.

The nucleotide coding sequences for the rtN236T, rtA181V or T mutations and the surrounding sequences from patients who developed these mutations are summarized in the following table (Table 3a). The corresponding changes in enzyme restriction sites are also listed in the table.

TABLE 3a

Restricti n Sit Changes
frm Wild-typ Baselin

| | | | | | Knock | |
|---|---|---|---|---|---|---|
| Patient ID | Study | Visit | Mutation | Nucleotide Sequences (5′ to 3′) | Out Site | Create Site |
| 0454-2506 | 438 | Baseline | wild-type | GGT ATA CAT TTA AAC CCG GAC AAA ACA | Dra I | Uba1442 I, |
| | | week 96 | rtN236T | GGT ATA CAT TTA ACC CCG GAC AAA ACA | | BsaJ I |
| 0626-1537 | 438 | Baseline | wild-type | GGT ATA CAT TTA AAC CCT AAC AAA ACA | Dra I | Cje I, HgiE |
| | | week 96 | rtN236T | GGT ATA CAT TTA ACC CCT ACC AAA ACA | | II |
| 0474-5508 | 438 | Baseline | wild-type | GGC ATA CAT TTA AAC CCT AAC AAA ACM | Dra I | None |
| | | week 144 | rtN236T | GGC ATA CAT TTA ACC CCT AAC AAA ACA | | |
| 0593-2027 | 435 | Baseline | wild-type | GGT ATA CAT CTA AAC CCT AAC AAA ACA | None | None |
| | | week 96 | rtN236T | GGT ATA CAT CTA ACC CCT AAC AAA ACA | | |
| 0585-2068 | 435 | Baseline | wild-type | GGT ATA CAT TTA AAC CCT ACT AAA ACT | Cje I, | Mn II |
| | | week 96 | rtN236T | GGT ATA CAT TTA ACC CCT CAC AAA ACA | Dra I, | |
| | | | | | BscH I | |
| 0341-2026 | 437 | Baseline | wild-type | GGT ATA CAT TTA AAC CCT AAT AAA ACC | Dra I | None |
| | | Month 38 | rtN236T | GGT ATA CAT TTA ACC CCT AAT AAA ACC | | |
| | | (Jan. 20, 2003) | | | | |
| 0624-1517 | 438 | Baseline | wild-type | CCG TTT CTC CTG GCT CAG TTT ACT AGT | Drd II | CviK I, |
| | | week 96 | rtA181V | CCG TTT CTC CTG GTT CAG TTT ACT AGT | | M.CviA IV, |
| | | | | | | Bst295 I, |
| | | | | | | CviJ I, CviT |
| | | | | | | I, BstDE I, |
| | | | | | | Dde I, BspCN |
| | | | | | | I |
| 0624-1564 | 438 | Baseline | wild-type | CCG TTT CTC CTG GCT CAG TTT ACT AGT | Drd II | Cvik I, |
| | | week 96 | rtA181V | CCG TTT CTC CTG GTT CAG TTT ACT AGT | | M.CviA IV, |
| | | | | | | Bst295 I, |
| | | | | | | CviJ I, CviT |
| | | | | | | I, BstDE I, |
| | | | | | | Dde I, BspCN |
| | | | | | | I |
| 0627-1557 | 438 | Baseline | wild-type | CCG TTT CTC CTG GCT CAG TTT ACT AGT | Drd II | CviK I, |
| | | week 144 | rtA181V | CCG TTT CTC CTG GTT CAG TTT ACT AGT | | M.CviA IV, |
| | | | | | | Bst295 I, |
| | | | | | | CviJ I, CviT |
| | | | | | | I, BstDE I, |
| | | | | | | Dde I, BspCN |
| | | | | | | I |
| 0454-2506 | 438 | Baseline | wild-type | CCG TTT CTC CTG GCT CAG TTT ACT AGT | Acc38 I, | Mly I, Ple I, |
| | | week 96 | rtA181T | CCG TTT CTC CTG ACT CAG TTT ACT AGT | BsaC I, | Sch I, Hpy188 |
| | | | | | BssK I, | III, BsmE I, |
| | | | | | Dsa V, | CviC I, |
| | | | | | EcoR II, | M.CcrM I, Hha |
| | | | | | M.EcoDcm, | II, Hinf I, |
| | | | | | M.Nla X, | Bst295 I, |
| | | | | | PspG I, | BstDE I, Dde |
| | | | | | BstN I, | I, BspCN I |
| | | | | | BstO I, | |
| | | | | | Drd II, | |
| | | | | | ScrF I | |

5.1.2.1. Conserved Site Mutations

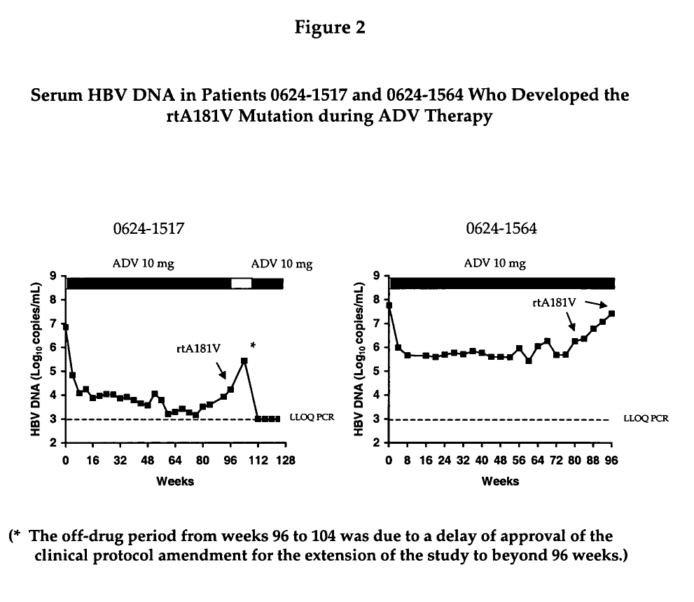

Five patients participating in study 438 developed mutations at conserved sites in the RT domain of HBV polymerase at week 96 (Table 3). The rtN236T mutation in the D domain of the HBV polymerase was observed in two patients: 0626-1537 and 0454-2506. In addition, a second conserved site mutation rtA181T was observed in conjunction with the rtN236T mutation in patient 0454-2506. Retrospective sequencing analysis for HBV isolates at earlier time points demonstrated that the rtN236T mutation became detectable at week 56 and week 80 in patients 0626-1537 and 0454-2506, respectively. The rtA181T mutation was only detected as a mixture with wild-type HBV in the week 96 sample from patient 0454-2506. The rtA181V mutation in the B domain of the HBV polymerase was separately observed in two patients (0624-1517 and 0624-1564) at week 96 and week 80, respectively (FIG. 2). Patient 0370-3503 developed double conserved site mutations rtK241E+rtK318Q at week 96 (Table 3).

### In Vitro and in Vivo Drug Susceptibility of the Conserved Site Mutations

To investigate if these conserved site mutations conferred resistance to adefovir, adefovir susceptibility of HBV isolates derived from the above five patients was evaluated using a whole HBV genome cell culture assay as described above.

rtN236T Mutation

In vitro phenotypic analysis of HBV clones derived from patients 0626-1537 and 0454-2506 demonstrated that the rtN236T mutation conferred a 7- to 14-fold decrease in adefovir susceptibility when compared the week 96 HBV clones containing the rtN236T mutation to the baseline wild-type clones (Table 4).

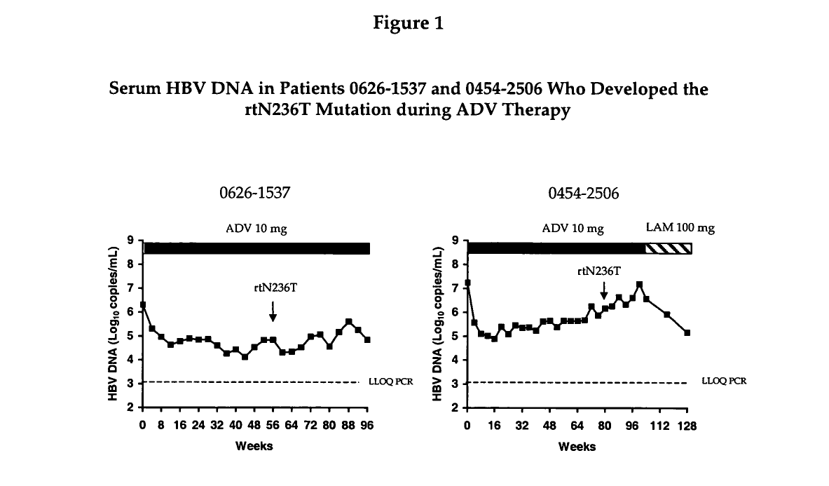

Both patients who developed the rtN236T mutation showed sub-optimal serum HBV DNA response after the initiation of ADV therapy with serum HBV reduced by less than 2 log₁₀ copies/mL from baseline (FIG. 1). After the modest initial responses, serum HBV DNA levels in these two patients gradually increased toward the baseline levels during ADV treatment (FIG. 1). Emergence of the rtN236T mutation was associated with a transient ALT flare (451 IU/L at week 92) in patient 0454-2506 but not in patient 0626-1537. The in vitro and clinical data confirmed that the rtN236T mutation was associated with reduced susceptibility to adefovir.

TABLE 4
In Vitro Adefovir Susceptibility of HBV Isolates from
Patients Who Developed Conserved Site Mutations
at Week 96 in Study 438

| | Mutation at | Adefovir IC₅₀ (μM) | | IC₅₀ Fold Change |
|---|---|---|---|---|
| Patient ID | Week 96 | Baseline | Week 96 | (Week96/Baseline |
| 0626-1537 | rtN236T | 0.26 ± 0.17 | 3.64 ± 2.78 | 13.8 |
| 0454-2506 | rtN236T¹ | 0.21 ± 0.01 | 1.55 ± 0.91 | 7.3 |
| 0624-1517 | rtA181V | 0.21 ± 0.07 | 0.52 ± 0.11 | 2.5 |
| 0624-1564 | rtA181V | 0.21 ± 0.01 | 0.62 ± 0.29 | 3.0 |
| 0370-3503 | rtK241E + | 0.16 ± 0.01 | 0.14 ± 0.03 | 0.9 |
| | rtK318Q | | | |

¹Both rtN236T and rtA181T mutations were observed in this patient at week 96. Patient-derived clones containing both rtN236T and rtA181T from the week 96 serum were replication defective in vitro; only clones with the single rtN236T mutation were tested.

rtA181T Mutation

One patient (0454-2506) with the rtN236T mutation also developed a second conserved site mutation rtA181T at week 96. However, all week 96 isolates derived from the patient encoding both rtN236T and rtA181T were replication deficient in vitro; the reason for this is unclear. To assess the adefovir sensitivity of HBV containing both rtN236T and rtA181Tmutations, an artificial construct was obtained by modifying an existing patient-derived HBV clone. The rtA181T mutation was introduced by site-directed mutagenesis into replicating HBV clones from patient 0454-2506 that already encoded rtN236T. The resulting double mutants were replication competent and were used for adefovir susceptibility testing. Unexpectedly, susceptibility of the rtN236T+rtA181T double mutant to adefovir was partially restored (2.5-fold resistant) compared to the single rtN236T mutant (Table 5). The rtA181T mutation was also introduced into a standard HBV lab strain (genotyped, ayw) to assess the individual contribution of the rtA181T mutation. The rtA181T mutant lab strain remained susceptible to adefovir with IC₅₀ changed by only 1.3-fold in vitro (Table 5). The in vitro phenotyping data suggested that rtA181T does not confer resistance to adefovir, but would have utility in HBV diagnostics.

TABLE 5
In Vitro Adefovir Susceptibility of HBV Strains
Containing the rtA181T Mutation

| | | Adefovir IC₅₀ | IC₅₀ Fold Change |
|---|---|---|---|
| HBV | Mutation | (μM) | from Wild-Type |
| Patient-derived | Wild-type (baseline) | 0.21 ± 0.01 | 1 |
| HBV | rtN236T (week 96) | 1.55 ± 0.91 | 7.3 |
| (0454-2506) | rtN236T + | 0.53 ± 0.26 | 2.5 |
| | rtA181T (week 96) | | |
| Lab HBV | Wild-type | 0.19 ± 0.03 | 1 |
| strain | rtA181T | 0.24 ± 0.03 | 1.3 |

rtA181V Mutation

Week 96 HBV clones derived from two patients (0624-1517 and 0624-1564) who developed the rtA181V mutant HBV exhibited a reproducible 2.5- to 3-fold reduction in adefovir susceptibility in vitro (Table 4). The rtA181V mutation appears to confer a low degree of reduced susceptibility to adefovir relative to the adefovir-resistant rtN236T mutation (7- to 14-fold resistance to adefovir in vitro). In addition, the two patients with the rtA181V mutation displayed inconsistent clinical profiles. Patient 0624-1564 had a rebound in serum HBV DNA to baseline by week 96 (FIG. 2), however, this patient also frequently missed ADV tablets during the clinical study. In contrast, serum HBV DNA in the other patient (0624-1517) remained suppressed below 1000 copies/mL after a period of treatment interruption during which this mutation was detected (FIG. 2). ALT levels did not change in either of these patients. The clinical significance of the rtA181V mutation is unclear based on the in vitro drug susceptibility data and the disparate clinical profiles of the two patients, although as noted it would have diagnostic utility for HBV infection.

rtK214E and rtK318O Double Mutations

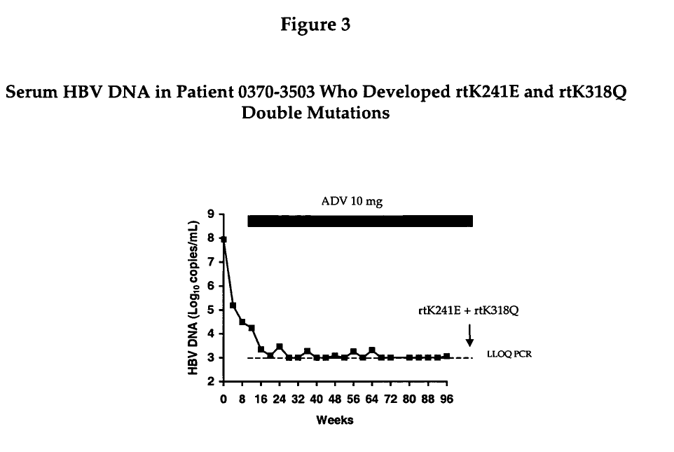

One patient (0370-3503) developed two conserved site mutations (rtK214E and rtK318Q) at week 96 in study 438. In vitro phenotypic analysis of HBV isolates from this patient demonstrated that the rtK241E+rtK318Q mutant HBV remained fully susceptible to adefovir with the IC₅₀ of adefovir changed by less than 0.9-fold compared to the baseline wild-type HBV clones (Table 4). This patient achieved a >5 log₁₀ reduction in serum HBV DNA to 3.1 log₁₀ copies/mL at week 96 with no evidence of serum HBV DNA rebound (FIG. 3). The latest available HBV DNA data (3.6 logl₁₀ copies/mL) as of February of 2003 (week 156) showed that the serum HBV DNA remains durably suppressed. The double mutation rtK241E+rtK318Q is not associated with adefovir resistance in vitro or clinically, but would be of value in HBV diagnosis.

Cross-resistance

Cross-resistance of rtN236T and rtA181V to lamivudine was tested in vitro (Table 6). HBV isolates with either the rtN236T or rtA181V mutations exhibited 2.3 to 3.5-fold decreases in lamivudine susceptibility, suggesting that these mutations may not cause clinical failure of lamivudine. In addition, one patient with the rtN236T mutation (0454-2506) withdrew from the clinical study and switched to lamivudine monotherapy at week 104 and achieved undetectable serum HBV DNA by the Digene assay (detection limit =1.5×10⁵ copies/mL) as well as ALT normalization after 6 months (FIG. 1) [18]. The clinical serum HBV DNA response to lamivudine in this patient further confirmed the in vitro finding that the rtN236T mutation did not confer significant cross-resistance to lamivudine.

Cross-resistance of the rtN236T mutation to acydic nucleotide analogs tenofovir and MCC-478 (free acid form) and nudeoside analogs L-dT and entecavir was also tested in vitro (Table 7). The rtN236T mutation demonstrated moderate cross-resistance (4- to 8-fold) to acyclic nucleotides tenofovir and MCC-478 in vitro. However, the rtN236T mutant remained susceptible to nucleoside analogs L-dT and entecavir in vitro, suggesting that patients infected with adefovir-resistant HBV may be treated with L-dT or entecavir.

TABLE 6
In Vitro Lamivudine Susceptibility of HBV Isolates
from Patients Who Developed Conserved Site
Mutations at Week 96 in Study 438

| | Mutation | | | |
|---|---|---|---|---|
| | at | Lamivudine IC₅₀ (μM) | | IC₅₀ Fold Change |
| Patient ID | Week 96 | Baseline | Week 96 | (Wk96/Baseline) |
| 0626-1537 | rtN236T | 0.035 ± 0.010 | 0.12 ± 0.06 | 3.5 |
| 0454-2506 | rtN236T¹ | 0.031 ± 0.016 | 0.070± | 2.3 |
| 0624-1517 | rtA181V | 0.070 ± 0.040 | 0.21 ± 0.08 | 3.0 |
| 0624-1564 | rtA181V | 0.046 ± 0.001 | 0.141± | 3.1 |
| 0370-3503 | rtK241E + | NA² | NA² | NA² |
| | rtK318Q | | | |

¹Both rtN236T and rtA181T mutations were observed in this patient at week 96. Patient-derived clones containing both rtN236T and rtA181T from week 96 serum were replication defective in vitro; only clones with the single rtN236T mutation were tested.
²Not analyzed.

TABLE 7
In Vitro Drug Susceptibility of a Patient-Derived HBV
Strain that Carries the rtN236T Mutation

| | IC₅₀ (μM) | | IC₅₀ Fold Change |
|---|---|---|---|
| Compounds | Wild-type | rtN23T Mutant | (rtN236T/Wild-type) |
| Adefovir | 0.15 | 1.47 | 9.6 |
| Tenofovir | 0.13 | 0.55 | 4.2 |
| MCC-478¹ | 0.030 | 0.25 | 8.6 |
| Lamivudine | 0.031 | 0.070 | 2.3 |
| L-dT | 0.14 | 0.33 | 2.4 |
| Entecavir | 0.00039 | 0.00026 | 0.67 |

¹Free acid of MCC-478

Accordingly, patients with resistance mutations optionally treated by one or more anti-HBV therapeutics that are not cross resistant, most notably tenofovir, MCC-478, lamivudine, L-dT or entecavir. Typically, the treatment is by coadministration (either as a single, coformulated dosage form such as a tablet or by coadministration in a course of therapy). Generally, two agents are employed together, e.g. tenofovir and adefovir, entcavir and adefovir, L-dT and adefovir, lamivudine and adefovir and MCC-478 and adefovir.

### Effect of the rtN236T, rtA181V and rtA181T mutations on HbsAg

The genome of HBV is organized into overlapping reading frames. The HBV surface antigen (HBsAg) gene is completely overlapped by the HBV polymerase gene. The HBV polymerase mutations (rtV173L, rtL180M, and rtM204V or I) associated with lamivudine resistance simultaneously cause HBsAg mutations that confer reduced binding affinity to anti-HBsAg antibody from vaccine sera [19]. These findings raise the possibility that lamivudine-selected HBsAg mutations may have the potential to escape neutralization by vaccine induced anti-HBsAg antibody. In contrast to the lamivudine resistance mutations, the adefovir resistance mutation rtN236T is located downstream of the stop codon of the HBsAg gene. Consequently, the rtN236T mutation does not cause any change in the HBsAg protein and, thus, has no risk of becoming a vaccine escape variant. However, the rtA181V and rtA181T mutations in the HBV reverse transcriptase do simultaneously cause mutations in the HBsAg. The rtA181V mutation causes a sL173F mutation in the HBsAg while the rtA181T mutation causes a stop codon in the HBsAg open-reading frame. The clinical significance of these corresponding HBsAg mutations remains unclear. In an embodiment of the invention, surface antigen bearing the sL173F mutation or terminating immediately before L172 is diagnostically useful for determining emergence of the mutations or for preparing vaccines useful in therapy of these mutations.

All references and citations herein are expressly incorporated by reference.

References

1. Lai C-L, Dienstag J, Schiff E, Leung N W Y, Atkins M, Hunt C, Brown N, Woessner M, Boehme R and Condreay L. Prevalence and clinical correlates of YMDD variants during lamivudine therapy for patients with chronic hepatitis B. Clinical Infectious Diseases 2003;36:687-696

2. Stuyver L J, Locarnini S A, Lok A, Richman D D, Carman W F, Dienstag J L and Schinazi R F. Nomenclature for antiviral-resistant human hepatitis B virus mutations in the polymerase region. Hepatology 2001;33:751-757

3. Delaney W E, Yang H, Westland C, Das K, Arnold E, Miller M, Gibbs C and Xiong S. Functional analysis of rtV173L, an HBV polymerase mutation frequently observed in lamivudine-resistant chronic hepatitis B patients [poster]. In: 53rd Annual Meeting of The American Association for the Study of Liver Diseases—The Liver Meeting. Boston, Mass: Poster 102710, 2002

4. Xiong X, Flores C, Yang H, Toole J J and Gibbs C S. Mutations in hepatitis B DNA polymerase associated with resistance to lamivudine do not confer resistance to adefovir in vitro. Hepatology 1998;28:1669-1673

5. Yokota T, Konno K, Shigeta S, Holý A, Balzarini J and De Clercq E. Inhibitory effects of acyclic nucleoside phosphonate analogues of hepatitis B virus DNA synthesis in HB611 cells. Antiviral Chemistry and Chemotherapy 1994;5:57-63

6. Yokota T, Mochizuki S, Konno K, Mori S, Shigeta S and De Clercq E. Inhibitory effects of selected antiviral compounds on human hepatitis B virus DNA synthesis. Antimicrobial Agents and Chemotherapy 1991;35:394-397

7. Heijtink R A, Kruining J, de Wilde G A, Balzarini J, De Clercq E and Schalm S W. Inhibitory effects of acyclic nucleoside phosphonates on human hepatitis B virus and duck hepatitis B virus infections in tissue culture. Antimicrobial Agents and Chemotherapy 1994;38:2180-2182

8. Heijtink R A, De Wilde G A, Kruining J, Berk L, Balzarini J, De Clercq E, Holý A and Schalm S W. Inhibitory effect of 9-(2-phosphonylmethoxyethyl)adenine (PMEA) on human and duck hepatitis B virus infection. Antiviral Research 1993;21:141-153

9. Korba B E, Milman G. A cell culture assay for compounds which inhibit hepatitis B virus replication. Antiviral Research 1991;15:217-228

10. Dandri M, Burda M M. Increased hepatocyte turnover and inhibition of woodchuck hepatitis B virus replication by adefovir in vitro do not lead to reduction of the closed circular DNA. Hepatology 2000;32:139-146

11. Marcellin P, Chang T-T, Lim S G, Tong M J, Sievert W, Shiffman M L, Jeffers L, Goodman Z, Wulfsohn M S, Xiong S, Fry J, Brosgart C and for the Adefovir Dipivoxil 437 Study Group. Adefovir dipivoxil for the treatment of hepatitis B e antigen-positive chronic hepatitis B. New England Journal of Medicine 2003;348:808-816

12. Hadziyannis S J, Tassopoulos N, Heathcote E J, Chang T-T, Kitis G, Rizzetto M, Marcellin P, Lim S G, Goodman Z, Wulfsohn M S, Xiong S, Fry J, Brosgart C and for the Adefovir Dipivoxil 438 Study Group. Adefovir dipivoxil for the treatment of hepatitis B e antigen-negative chronic hepatitis B. New England Journal of Medicine 2003;348:800-807

13. Hadziyannis S, Tassopoulos N, Heathcote J, Chang T T, Kitis G, Rizetto M, Marcellin P, Lim S G, Chen S-S, Wulfsohn M, Wollman M, Fry J, Brosgart C and 438 Study Group. Two year results from a double-blind, randomnized, placebo-controlled study of adefovir dipivoxil (ADV) for presumed precore mutant chronic hepatitis B [abstract]. Journal of Hepatology 2003;38:143. Abstract 492

14. Westland C E, Yang H, Delaney W E, IV, Gibbs C S, Miller M D, Wulfsohn M, Fry J, Brosgart C L, Xiong S and the 437 and 438 Study Teams. Week 48 resistance surveillance in two phase 3 clinical studies of adefovir dipivoxil for chronic hepatitis B. Hepatology 2003;38:96-103

15. Benhamou Y, Bochet M, Thibault V, Calvez V, Fievet M H, Vig P, Gibbs C S, Brosgart C, Fry J, Namini H and Katlama C. Safety and efficacy of adefovir dipivoxil in patients co-infected with HIV-1 and lamivudine-resistant hepatitis B virus: an open-label pilot study. Lancet 2001;358:718-723

16. Westland C E, Delaney W, IV, Yang H, Gibbs C S, Miller M D, Lama N, Fry J, Brosgart C L, Wulfsohn M and Xiong S. Genotypic analysis of HBV isolates from liver transplant patients with lamivudine-resistant HBV who received adefovir dipivoxil therapy for one year [abstract]. In: Therapies for Viral Hepatitis. Boston, Mass: Abstract 2748, 2002

17. EASL Jury. EASL International Consensus Conference on Hepatitis B. 13-14 Sep., 2002: Geneva, Switzerland. Consensus statement (short version). Journal of Hepatology 2003;38:533-540

18. Angus P, Vaughan R, Xiong S, Yang H, Delaney W, Gibbs C, Brosgart C, Colledge D, Edwards R, Ayres A, Bartholomeusz A and Locarnini S. Resistance to adefovir dipivoxil therapy associated with the selection of a novel mutation in the HBV polymerase. Gastroenterology 2003;125:292-297

19. Torresi J. The virological and clinical significance of mutations in the overlapping envelope and polymerase genes of hepatitis B virus. Journal of Clinical Virology 2002;25:97-106

We claim:

1. Isolated nucleic acid encoding hepatitis B virus rtA181V or rtA181T, or their complementary nucleic acids.

2. The nucleic acid of claim 1 which is human hepatitis B virus.

3. The nucleic acid of claim 2 which is intact infectious virus.

4. The nucleic acid of claim 2 which is fused to heterologous nucleic acid.

5. The nucleic acid of claim 2 which is about from 10 to 35 base pairs.

6. Duck hepatitis B virus rtA181V or rtA181T.

7. A duck infected with duck hepatitis B virus rtA181V or rtA181T

8. Woodchuck hepatitis virus rtA181V or rtA181T.

9. A woodchuck infected with woodchuck hepatitis virus rtA181V or rtA181T.

10. A vector comprising the nucleic acid of claim 1.

11. A host cell transformed with a vector of claim 10.

12. A method comprising culturing a host cell of claim 11 and recovering rtA181V or rtA181T therefrom.

13. A reverse transcriptase comprising (a) isolated hepatitis B virus rtA181V or rtA181T and/or (b) rtA181V and/or rtA181T fused to a heterologous polypeptide.

14. The reverse transcriptase of claim 13 bound to a detectable label, bound to an insoluble substance, or formulated in a pharmaceutically acceptable excipient.

15. The isolated reverse transcriptase of claim 13 in an infectious hepatitis B virus.

16. An antibody capable of specifically binding rtA181V or rtA181T.

17. The antibody of claim 15 bound to a detectable label, bound to an insoluble substance or formulated in a pharmaceutically acceptable excipient.

18. A method for immunotherapy comprising administering to a subject the isolated reverse transcriptase of claim 13.

19. A method for immunotherapy comprising administering to a subject the antibody of claim 16.

20. A method for the treatment of HBV comprising administering adefovir to a subject infected with HBV, determining whether the subject is infected with HBV rtA181V or rtA181T and, if so, administering to the subject a non-cross reactive anti-HBV drug in addition adefovir.

21. The method of claim 20 wherein the adefovir and the drug are administered substantially simultaneously to the subject.

22. The method of claim 20 wherein the drug is selected from the group consisting of entecavir, L-dT, MCC-478, FTC, L-dC, L-FMAU, L-Fd4C, Lamivudine and tenofovir.

23. A method for the prevention of emergence of rtA181V or rtA181T in a subject undergoing therapy for HBV comprising administering adefovir and at least one non-cross reactive anti-HBV drug.

24. The method of claim 23 wherein adefovir and the anti-HBV drug are administered substantially simultaneously.

25. A diagnostic PCR kit for HBV rtA181V or rtA181T comprising primers capable of specifically amplifying an HBV rt sequence containing rtA181V or rtA181T.

26. Isolated nucleic acid encoding hepatitis B virus reverse transcriptase sL173F or HBV sL172trunc, or their complementary sequences.

27. The nucleic acid of claim 26 which is human hepatitis B virus.

28. The nucleic acid of claim 27 which is intact virus.

29. The nucleic acid of claim 27 which is fused to a heterologous nucleic acid.

30. The nucleic acid of claim 27 which is about from 10 to 35 base pairs.

31. Duck hepatitis B virus sL173F or sL172trunc.

32. A duck infected with duck hepatitis B virus sL173F or sL172trunc.

33. Woodchuck hepatitis virus sL173F or sAg truncated just N-terminal to sL172F.

34. A woodchuck infected with woodchuck hepatitis virus sL173F or sL172trunc.

35. A vector comprising the nucleic acid of claim 26.

36. A host cell transformed with a vector of claim 35.

37. A method comprising culturing a host cell of claim 36 and recovering sL173F or sL172trunc therefrom.

38. A hepatitis B virus sAg comprising (a) isolated hepatitis B virus sL173F or sL172trunc and/or (b) sL173F or sL172trunc fused to a heterologous polypeptide.

39. The sAg of claim 38 bound to a detectable label, bound to an insoluble substance, or formulated in a pharmaceutically acceptable excipient.

40. The isolated sAg of claim 38 in an infectious hepatitis B virus.

41. An antibody capable of specifically binding sL173F or sL172trunc.

42. The antibody of claim 40 bound to a detectable label, bound to an insoluble substance or formulated in a pharmaceutically acceptable excipient.

43. A method for immunotherapy comprising administering to a subject the isolated sAg of claim 38.

44. A method for immunotherapy comprising administering to a subject the antibody of claim 41.

45. A method for the treatment of HBV comprising administering adefovir to a subject infected with HBV, determining whether the subject is infected with HBV sL173F or sL172trunc and, if so, additionally administering to the subject a non-cross reactive anti-HBV drug.

46. The method of claim 45 wherein the adefovir and the drug are administered substantially simultaneously to the subject.

47. The method of claim 45 wherein the drug is selected from the group consisting of entecavir, L-dT, MCC-478, FTC, L-dC, L-FMAU, L-Fd4C, Lamivudine and tenofovir.

48. A method for the prevention of emergence of HBV sL173F or sL172trunc in a subject undergoing therapy for HBV comprising administering adefovir and at least one non-cross reactive anti-HBV drug.

49. The method of claim 48 wherein adefovir and the anti-HBV drug are administered substantially simultaneously.

50. A diagnostic PCR kit for HBV sL173F or sL172trunc comprising primers capable of specifically amplifying an HBV rt sequence containing HBV sL173F or sL172trunc.


SEQUENCE LISTING (24 sequences)

SEQ ID NO: 1 - protein, 4 aa, Artificial Sequence
  Description of Artificial Sequence Synthetic motif sequence
YMDD

SEQ ID NO: 2 - protein, 5 aa, Artificial Sequence
  Description of Artificial Sequence Synthetic peptide
SVRFS

SEQ ID NO: 3 - DNA, 41 bp, Artificial Sequence
  Description of Artificial Sequence Synthetic primer
ccggaaagcttgagctcttctttttcacctctgcctaatca

SEQ ID NO: 4 - DNA, 40 bp, Artificial Sequence
  Description of Artificial Sequence Synthetic primer
ccggaaagcttgagctcttcaaaaagttgcatggtgctgg

SEQ ID NO: 5 - DNA, 27 bp, Artificial Sequence
  Description of Artificial Sequence Synthetic oligonucleotide
ggtatacatttaaacccggacaaaaca

SEQ ID NO: 6 - DNA, 27 bp, Artificial Sequence
  Description of Artificial Sequence Synthetic oligonucleotide
ggtatacatttaaccccggacaaaaca

SEQ ID NO: 7 - DNA, 27 bp, Artificial Sequence
  Description of Artificial Sequence Synthetic oligonucleotide
ggtatacatttaaaccctaacaaaaca

SEQ ID NO: 8 - DNA, 27 bp, Artificial Sequence
  Description of Artificial Sequence Synthetic oligonucleotide
ggtatacatttaacccctaccaaaaca

SEQ ID NO: 9 - DNA, 27 bp, Artificial Sequence
  Description of Artificial Sequence Synthetic oligonucleotide
ggcatacatttaaaccctaacaaaacm

SEQ ID NO: 10 - DNA, 27 bp, Artificial Sequence
  Description of Artificial Sequence Synthetic oligonucleotide
ggcatacatttaacccctaacaaaaca

SEQ ID NO: 11 - DNA, 27 bp, Artificial Sequence
  Description of Artificial Sequence Synthetic oligonucleotide
ggtatacatctaaaccctaacaaaaca

SEQ ID NO: 12 - DNA, 27 bp, Artificial Sequence
  Description of Artificial Sequence Synthetic oligonucleotide
ggtatacatctaacccctaacaaaaca

SEQ ID NO: 13 - DNA, 27 bp, Artificial Sequence
  Description of Artificial Sequence Synthetic oligonucleotide
ggtatacatttaaaccctactaaaact

SEQ ID NO: 14 - DNA, 27 bp, Artificial Sequence
  Description of Artificial Sequence Synthetic oligonucleotide
ggtatacatttaacccctcacaaaaca

SEQ ID NO: 15 - DNA, 27 bp, Artificial Sequence
  Description of Artificial Sequence Synthetic oligonucleotide
ggtatacatttaaaccctaataaaacc

SEQ ID NO: 16 - DNA, 27 bp, Artificial Sequence
  Description of Artificial Sequence Synthetic oligonucleotide
ggtatacatttaacccctaataaaacc

SEQ ID NO: 17 - DNA, 27 bp, Artificial Sequence
  Description of Artificial Sequence Synthetic oligonucleotide
ccgtttctcctggctcagtttactagt

SEQ ID NO: 18 - DNA, 27 bp, Artificial Sequence
  Description of Artificial Sequence Synthetic oligonucleotide
ccgtttctcctggttcagtttactagt

SEQ ID NO: 19 - DNA, 27 bp, Artificial Sequence
  Description of Artificial Sequence Synthetic oligonucleotide
ccgtttctcctggctcagtttactagt

SEQ ID NO: 20 - DNA, 27 bp, Artificial Sequence
  Description of Artificial Sequence Synthetic oligonucleotide
ccgtttctcctggttcagtttactagt

SEQ ID NO: 21 - DNA, 27 bp, Artificial Sequence
  Description of Artificial Sequence Synthetic oligonucleotide
ccgtttctcctggctcagtttactagt

SEQ ID NO: 22 - DNA, 27 bp, Artificial Sequence
  Description of Artificial Sequence Synthetic oligonucleotide
ccgtttctcctggttcagtttactagt

SEQ ID NO: 23 - DNA, 27 bp, Artificial Sequence
  Description of Artificial Sequence Synthetic oligonucleotide
ccgtttctcctggctcagtttactagt

SEQ ID NO: 24 - DNA, 27 bp, Artificial Sequence
  Description of Artificial Sequence Synthetic oligonucleotide
ccgtttctcctgactcagtttactagt